(19) United States
(12) Patent Application Publication
(10) Pub. No.: US 2022/0335477 A1
(43) Pub. Date: Oct. 20, 2022

(54) METHOD AND SYSTEM FOR DISTRIBUTING DIGITAL MEDIA DEPENDING ON MOBILITY PARAMETERS OF AN INDIVIDUAL

(71) Applicant: ZHOR TECH, Nancy (FR)

(72) Inventor: Karim OUMNIA, Nancy (FR)

(21) Appl. No.: 17/763,281

(22) PCT Filed: Sep. 24, 2020
(86) PCT No.: PCT/EP2020/076820

§ 371 Date: May 17, 2022

(30) Foreign Application Priority Data
Sep. 24, 2019 (EP) 19306194.2
Apr. 14, 2020 (EP) 20169505.3

(51) Int. Cl. G06Q 30/02

(52) U.S. Cl. CPC G06Q 30/0271; G06Q 30/0255

(57) ABSTRACT

The invention relates to a digital media distribution method and a digital media distribution system (1) configured to distribute digital media (301), characterized in that said digital media (301) are distributed to presentation computing devices (20) each associated with at least one connected sole (10), said digital media distribution system comprising a calculation computing device (30) configured to load (510) mobility or posture parameter values (101) calculated from raw data generated by at least one connected sole (10); Load (520) shoe parameter values (201); Load (530) digital media (301) and a distribution repository (311) including digital media (301) distribution rules (322); Transmit (540) personalized digital media (401) to at least one presentation computing device (20) coupled to the at least one connected sole (10) having generated said raw data.

The invention relates to the field of the distribution of digital media, more particularly of personalized digital media, according to mobility parameters of an individual. The invention relates to a digital media distribution system configured to distribute digital media, according to the distribution rules, to computing devices coupled to connected soles. The invention further relates to a computing device, which is preferably mobile, coupled to at least one connected sole and configured to receive digital media according to distribution rules and mobility parameters.

### PRIOR ART

During the last century, the evolution of fashion, the development of production methods in the field of textiles or more generally ready-to-wear and information and communication technologies have contributed to the democratization of access to clothing, that is to say textiles and footwear items, by a large part of the world's population. Today, the consumer is faced with a variety of choices and selections that were not available before. To facilitate the dissemination of this wide choice to consumers, many solutions have emerged, mainly developed by major clothing brands or digital giants. These solutions aim in particular at identifying the web pages visited by consumers in order to offer them appropriate and suggestive content in the form of advertising media. This suggestion of products and services can be done via dedicated advertising inserts that can be found on digital platforms such as social network platforms or search engines. Thus, by identifying consumer behavior, it is possible to offer them personalized media in line with the needs potentially expressed by consumers. More particularly, in the field of sport, the choice of clothing or footwear items is of paramount importance. Indeed, at the beginning of the last century, a consumer wishing to practice a particular sporting discipline would have had access to only a limited choice of sportswear.

Today, each brand or clothing seller offers a significant quantity of items. Thus, the consumer wishing to equip himself with clothing adapted to his everyday life or to the practice of a particular sporting discipline may no longer know which choice is the most appropriate with regard to his activity and his own characteristics. This difficulty in choosing suitable clothing is above all taken into consideration in the current technical solutions for footwear items. For example, in order to provide all the necessary information to a consumer wishing to obtain footwear items, a solution, described in patent application U.S. Ser. No. 11/009,605, consists in identifying the technical characteristics of each footwear item allowing the consumer to identify one or more footwear items that meet their needs. This solution in particular proposes a method for supplying shoes in a retail store, comprising steps consisting in supplying a packaging for shoes displaying values for a plurality of performance characteristics, in particular stability, damping and adjustment associated with one or more footwear items. This allows the consumer to deliver particularly relevant information to consumers wishing to obtain footwear items adapted to a specific practice of a sporting activity. On the other hand, the consumer is still obliged to consult the information, delivered by the display screen, and which has the technical characteristics of all the footwear items offered, which can constitute a difficulty as regards the choice of the footwear item the more suited to the personal practice of a sport by the consumer. In addition, the consumer can only access this information by going directly to the sign offering this type of display. Furthermore, digital media management, more particularly targeting consumers with an interest in the content of a given digital media, often has challenges. Indeed, generally digital media are sent to a consumer from known information, for example concerning a previous purchase or via “cookies” whose well-known function is to store the behavioral data of users/consumers navigating on websites and are at the heart of targeted advertising devices on the web. For example, when a consumer browses several shoe sites, the latter will be confronted with advertisements on the subject of the shoe thereafter because of the data contained in the cookies. Very often, the consumer finds himself confronted with sending a myriad of digital media, which can lead to saturation of the data transfer servers or require more computer resource requirements to process all the digital media by processing servers. Moreover, in order to overcome the saturation of data transfer servers, digital media often contain a maximum of references (to a plurality of footwear items for example), the relevant information, which could be contained in the digital media, is thus lost in the middle of other information which is of little interest to the consumer. Finally, these solutions are generally deployed for footwear items, whereas the consumer's need is not limited to these footwear items alone.

Thus, the methods of the prior art are only based on indirect and occasional analyzes of user needs. Furthermore, the information collected is generally fragmented and does not allow to contextualize said information. There is therefore a need for new solutions allowing to offer truly personalized articles to a consumer.

### Technical Problem

The purpose of the invention is therefore to overcome the disadvantages of the prior art. In particular, the purpose of the invention is to provide a digital media distribution system configured to deliver personalized digital media directly to a consumer's electronic device that is able to take consumer activity into consideration when establishing digital media to be distributed. Furthermore, the invention relates to a method for distributing digital media capable of targeting digital media in the best way possible without invasively questioning the lifestyle of the targeted consumer. Furthermore, while managing digital media and sending it to users may result in possible saturation of data transfer servers or the need to commit resources to processing servers, this allows to reduce server load.

The invention further relates to a computing device, which is preferably mobile, configured to receive personalized digital media.

### BRIEF DESCRIPTION OF THE INVENTION

To this end, the invention relates to a digital media distribution system configured to distribute digital media according to distribution rules, characterized in that said digital media are distributed to presentation computing devices each associated with at least one connected sole, said at least one connected sole being associated with a footwear item, said digital media distribution system comprising a calculation computing device configured to:
Load mobility or posture parameter values calculated from raw data generated by the at least one connected sole, said mobility or posture parameter values being associated with one or more presentation computing devices, which are preferably mobile, each being associated with the at least one connected sole;
Load shoe parameter values, said shoe parameter values each relating to a footwear item associated with the at least one connected sole;
Load the digital media and a distribution repository including digital media distribution rules, preferably the digital media and the distribution repository having been sent by a media computing device; and
Transmit personalized digital media to at least one of the presentation computing devices associated with the at least one connected sole having generated said raw data from which the mobility or posture parameter values have been generated, said transmitted personalized digital media being selected, within the loaded digital media, according to the distribution rules, the mobility or posture parameter values and the shoe parameter values of the footwear item associated with the at least one connected sole.

According to another embodiment, a digital media distribution system is configured to distribute digital media according to distribution rules, characterized in that said digital media are distributed to at least one presentation computing device associated with at least one connected sole configured to generate raw use data, said at least one connected sole being associated with a footwear item, said digital media distribution system comprising a calculation computing device configured to:
Obtain mobility or posture parameter values calculated from raw use data;
Obtain shoe parameter values, said shoe parameter values relating to a footwear item associated with the at least one connected sole;
Obtain the digital media and a distribution repository including the digital media distribution rules, preferably the digital media and the distribution repository having been sent by a media computing device; and
Transmit personalized digital media to the at least one presentation computing device associated with the at least one connected sole having generated said raw use data from which the mobility or posture parameter values have been calculated, said transmitted personalized digital media being selected, within the loaded digital media, according to the distribution rules, of the mobility or posture parameter values and of the shoe parameter values of the footwear item associated with the at least one connected sole.

Such digital media distribution systems allow to select or create then transmit personalized digital media on the basis in particular of mobility or posture parameters of a user. Thus, without realizing it and without having to take an active approach, the user will be able to transmit key elements allowing third-party structures to provide him with an offer and services that are truly adapted. Creating personalized media is a tedious and complex step for companies because it is difficult to determine what will really be suitable for a particular user. Thanks to the present invention, the distribution system allows to collect and process information so as to automatically create appropriate media, without prejudice and without biased analysis of behavioral habits. Furthermore, the mobility or posture parameter values may be calculated at least in part in a connected sole from raw data generated by the sole. In addition, these personalized digital media can be sent to a computing device used by the user of the connected soles so as to more easily reach their audience.

The automated analysis of the activity that the user makes of a footwear item is therefore placed at the center of the digital media distribution system according to the invention and allows to obtain hitherto inaccessible information for generating the personalized digital media. Furthermore, beyond a fine selection of the media to be sent according to several parameters, the performance of part of the calculations are carried out in the connected sole, possibly supplemented by the selection of the moment of sending these media allow to reduce the server resources necessary for the implementation of the distribution.

According to other characteristics of a digital media distribution system according to the invention, the latter may optionally include one or more of the following characteristics, alone or in combination:
the calculation computing device is configured to further load environmental parameter values, said environmental parameter values corresponding to environmental conditions measured during use of the footwear item associated with the at least one connected sole. This allows to benefit from a particularly relevant indicator in order to be able to offer personalized digital media according to the user's needs, thus indicating to him an unsuitable use in connection with the environmental conditions in which the user of the footwear item moves, likely to cause injury or reduce the performance of the user.
the calculation computing device is configured to identify a triggering event and to transmit the personalized digital media to the at least one presentation computing device when a triggering event has been identified. This allows to provide digital media according to an event determined by the computing device according to various parameters related to the connected sole. The transmitted digital media therefore takes into account the user's situation by indicating one or more footwear items of interest.
the calculation computing device is configured to further load user parameter values, said user parameter values corresponding in particular to morphological data of the user of the footwear item associated with the at least one connected sole.
the shoe parameter values or the mobility or posture parameter values further include a wear datum of the footwear item associated with the at least one connected sole. This allows to benefit from a particularly relevant indicator to be able to offer personalized digital media according to the user's needs, thus indicating to him a deterioration of the footwear item likely to generate an injury or reduce the performance of the user.
the calculation computing device is configured to receive raw data generated by the at least one connected sole or pre-processed data produced from raw data generated by the at least one connected sole and the calculation computing device is configured to calculate, from these raw data or pre-processed data, the mobility or posture parameter values. Thus, the calculation of the mobility or posture parameter values is here carried out on the calculation computing device and can therefore benefit from a higher calculation power and a centralization of the process. Preferably, part of the processing of the raw data can be carried out at the connected sole(s). The pre-processing can then correspond for example to data filtering such as frequency filtering or noise suppression. Thus, it is possible to increase the autonomy of the connected soles and reduce the calculation power required at the calculation computing device.
the calculation computing device is configured to receive the mobility or posture parameter values, said mobility or posture parameter values having been generated by the at least one connected sole or the one or more presentation computing devices associated with the at least one connected sole. In this case, the mobility or posture parameter values were calculated at the sole or at the computing device. This allows to reduce the calculation power necessary at the calculation computing device. As before, preferably, part of the processing of the raw data can be carried out at the connected sole(s) and the other part is then carried out at the computing device.
the at least one connected sole is directly or indirectly coupled to the presentation computing device. Thus, there is preferably a communication link between the computing device and the at least one connected sole.
the mobility or posture parameter values are calculated from raw data generated by two connected soles. The use of two connected soles allows to obtain more precise and informative mobility or posture parameter values for better personalized digital media.
the personalized digital media includes data derived from shoe parameter values and/or data derived from mobility or posture parameter values. This allows to generate more personalized digital media.
the personalized digital media includes data derived from shoe parameter values and/or data derived from mobility or posture parameter values as well as personal data on the user of the connected sole(s). This allows to generate more personalized digital media.
the shoe parameters include reference use parameters and in that the calculation computing device is further configured to identify unconventional uses, said identification including a comparison of the reference use parameters with use parameters calculated from mobility or posture parameter values.
the calculation computing device is further configured to calculate one or more trend values based on the identification of unconventional uses correlated with geographical parameters. Thus, it will be possible to generate personalized digital media including geographic parameter data.
It includes an anonymization server, said anonymization server being configured to:
   identify, from a sensitive data identification repository, data to be anonymized in a message including the mobility or posture parameter values or raw data generated by the at least one connected sole or pre-processed data produced from raw data generated by the at least one connected sole;
   generate, from an anonymization repository, anonymized data from previously identified data to be anonymized; and
   generate an anonymized message from the anonymized data and the message.

Thus, the distribution system allows to preserve the personal data of users of connected soles.
the calculation computing device is further configured to transmit, to the media computing device, a digital media transmission confirmation message to the presentation computing device, which is preferably mobile, said transmission confirmation message including an identifier associated with a presentation computing device, which is preferably mobile, and an identifier of the transmitted digital media. Thus, the distribution system allows to preserve the personal data of users of connected soles.
the calculation computing device is further configured to transmit, to the media computing device, a message of connection to the presentation computing device, which is preferably mobile, said connection message including an identifier associated with said presentation computing device and an identifier associated with a shoe parameter value. Thus, without having critical, personal or sensitive information, the media computing device is able to receive information on the distribution of personalized digital media.
the calculation computing device is further configured to generate production parameters relating to the manufacture of footwear items on the basis of determined association index values, shoe parameter values and the wear datum. The wear datum can come from the connected sole or from the presentation device(s) associated with the connected sole. The calculation computing device can in particular receive wear parameter values from several connected soles used in different geographical areas. Thus, it is possible to plan production cycles according to the use and even unexpressed needs of users.
The personalized digital media encodes one or more information in a format selected from the format: Text, images, video, sounds and their combinations.

According to another aspect, the present invention relates to a method for distributing digital media according to distribution rules, characterized in that said digital media are distributed to at least one presentation computing device associated with at least one connected sole configured to generate raw use data, said at least one connected sole being associated with a footwear item, said method including the following steps executed by one or more calculation computing devices:
Obtaining mobility or posture parameter values calculated from raw use data;
Obtaining shoe parameter values, said shoe parameter values relating to the footwear item associated with the at least one connected sole;
Obtaining the digital media and a distribution repository including the digital media distribution rules, preferably the digital media and the distribution repository having been sent by a media computing device; and
Transmitting personalized digital media to the at least one presentation computing device associated with the at least one connected sole having generated said raw use data from which the mobility or posture parameter values have been calculated, said transmitted personalized digital media being selected, within the loaded digital media, according to the distribution rules, of the mobility or posture parameter values and of the shoe parameter values of the footwear item associated with the at least one connected sole.

Other advantages and characteristics of the invention will appear upon reading the following description given by way of illustrative and non-limiting example, with reference to the appended Figures:

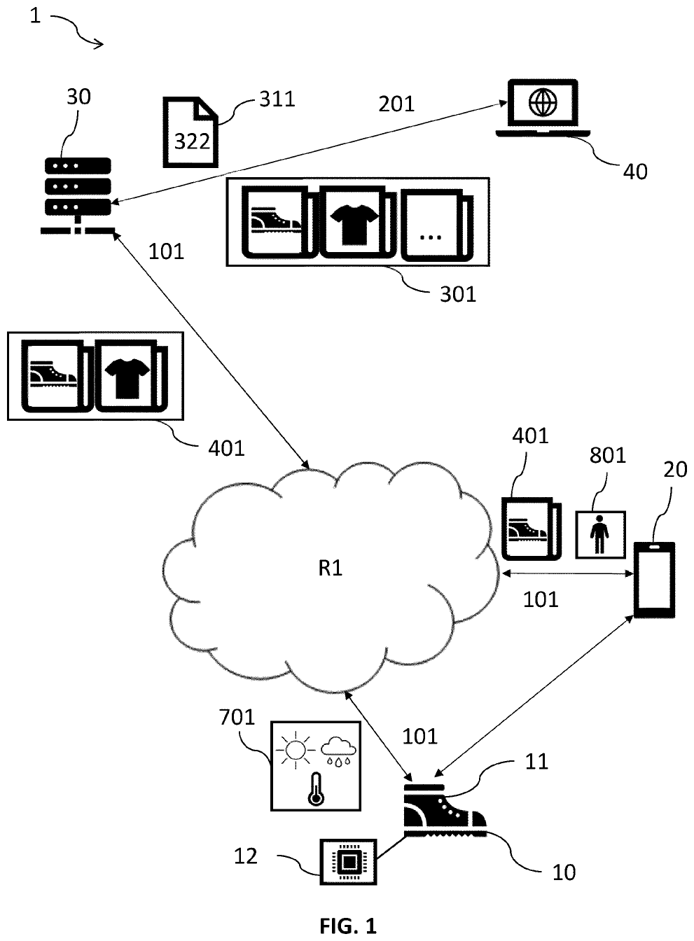

FIG. 1 shows an illustrative diagram of a digital media distribution system according to the invention.

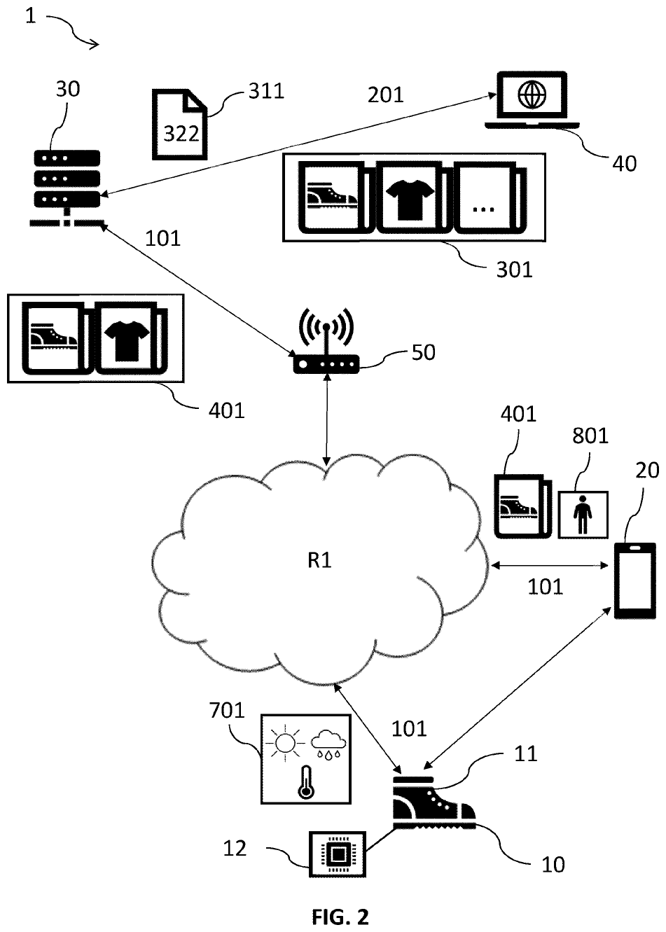

FIG. 2 shows an illustrative diagram of a digital media distribution system including an anonymization server in accordance with the invention.

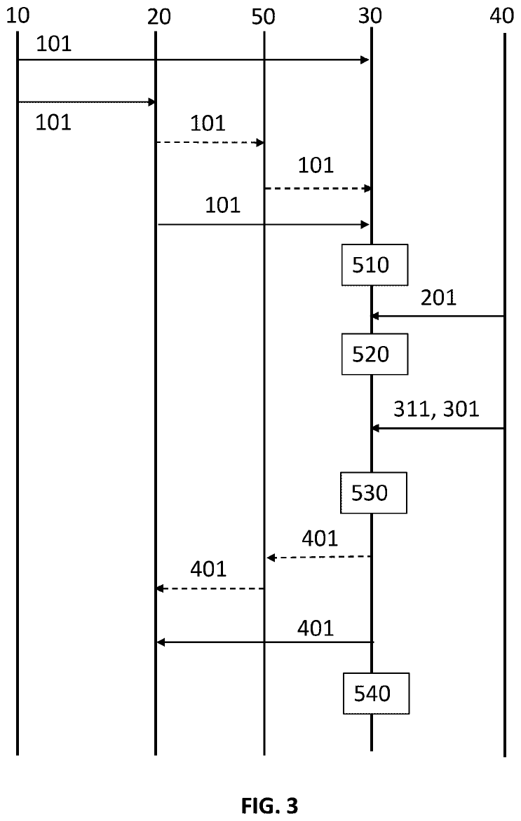

FIG. 3 shows a diagram illustrating the exchanges between the various devices of an embodiment of a digital media distribution system in accordance with the invention.

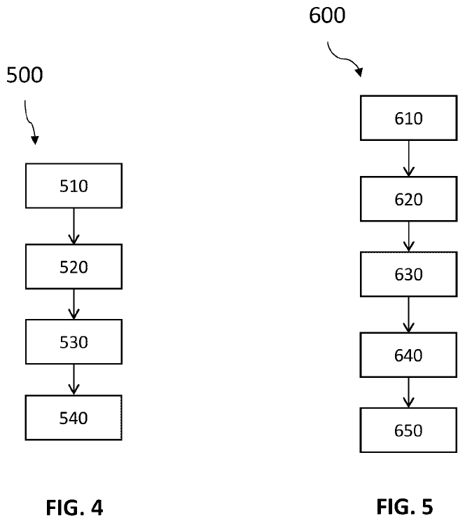

FIG. 4 shows an illustrative diagram of a digital media distribution method that can be implemented within a digital media distribution system in accordance with the invention.

FIG. 5 shows an illustrative diagram of a method for analyzing mobility or posture parameter values of a user for generating a message of possible change of category of a user, which can be implemented within a digital media distribution system according to the invention.

Aspects of the present invention are described with reference to flow charts and/or block diagrams of methods, apparatuses (systems), and computer program products according to embodiments of the invention.

In the figures, flowcharts and block diagrams illustrate the architecture, functionality, and operation of possible implementations of systems, methods, and computer program products according to various embodiments of the present invention. In this regard, each block in the flowcharts or block diagrams may represent a system, device, module, or code, which comprises one or more executable instructions to implement the specified logical function(s).

In some implementations, the functions associated with the blocks may appear in a different order than that shown in the figures. For example, two blocks shown in succession may, in fact, be executed substantially simultaneously, or the blocks may sometimes be executed in reverse order, depending on the functionality involved. Each block in the block diagrams and/or flowchart, and combinations of blocks in the block diagrams and/or flowchart, may be implemented by special hardware systems that execute the specified functions or acts or perform combinations of special equipment and computer instructions.

### DESCRIPTION OF THE INVENTION

In the remainder of the description, “posture”, “mobility” or “gait” within the meaning of the invention corresponds to the posture, movements, locomotion, and balance of the user.

Balance corresponds in particular to postural balance related to the stability of the body and more particularly to the stability of a user's center of gravity. Nevertheless, it can integrate both static and dynamic balance.

The expression “mobility or posture parameters” or “gait parameter” corresponds to biomechanical parameters identified in the static or dynamic position of a person, a characteristic of a person's posture or mobility. A biomechanical parameter can be determined by various calculation operations from parameter values generated by sensors of a connected sole.

“Digital media” means any digital communication medium containing information that can be delivered by a suitable electronic device. A digital media can thus correspond to a digital file encoding one or more data which, once decoded by a suitable computing device, allow to deliver said data in audio, image, video and/or text form.

The expression “movement analysis”, “mobility analysis” or “gait analysis” corresponds to the determination of a user's gait descriptor. It may correspond, within the meaning of the invention, to the attribution of one or more values, for example a score, a classification or a rating to a trajectory or to the displacement of a foot of a user. This characterization of the gait can allow to obtain one or more numerical or alphanumeric values of biomechanical parameters representative of the gait.

“Sole” means an object allowing to separate the user's foot from the ground. A shoe can include an upper sole layer in direct contact with the foot of the user and a lower sole layer in direct contact with the ground or more generally the external environment. A shoe may also include a removable inner sole.

“Removable” means the ability to be easily detached, removed or disassembled without having to destroy the fastening means either because there is no fastening means or because the fastening means are easily and quickly removable (e.g. notch, screw, tab, lug, clips). For example, by removable, it should be understood that the object is not fixed by welding or by any other means not intended to allow the object to be detached.

“Process”, “calculate”, “determine”, “display”, “transform”, “extract”, “compare” or more broadly “executable operation” mean, within the meaning of the invention, an action performed by a device or processor unless the context indicates otherwise. In this regard, operations refer to actions and/or processes of a data processing system, for example a computing system or an electronic computing device, which manipulates and transforms data represented as physical (electronic) quantities in computing system memories or other information storage, transmission or display devices. These operations can be based on applications or software.

The term “learning” within the meaning of the invention corresponds to a method designed to define one or more correspondences, which may or may not be in the form of a function f, allowing to calculate a value of Y from a base of n labeled (X1 . . . n, Y1 . . . n) or unlabeled (X1 . . . n) observations. Such a correspondence or function may correspond to a prediction model. Learning can be said to be supervised when it is based on labeled observations and unsupervised when it is based on unlabeled observations. In the context of the present invention, learning can advantageously be used to determine the personalized digital media to be transmitted, and possibly the instant of its transmission.

“Learning model” means any mathematical model that allows to analyze a volume of data and to establish relationships between factors allowing the assessment of risks or that of opportunities associated with a specific set of conditions, in order to guide decision-making towards a specific action.

“Computing device” means any device comprising a processing unit or a processor, for example in the form of a microcontroller cooperating with a data memory, possibly a program memory, said memories being able to be dissociated. The processing unit cooperates with said memories by means of an internal communication bus.

The terms or expressions “application”, “software”, “program code”, and “executable code” mean any expression, code or notation of a set of instructions intended to cause data processing to perform a particular function directly or indirectly (e.g. after a conversion operation to another code). The examples of program code may include, but is not limited to, a subroutine, function, an executable application, a source code, an object code, a library, and/or any other sequence of instructions designed for the running on a computing system.

Within the meaning of the invention, the term “processor” designates at least one hardware circuit configured to execute instructions contained in the program code. The hardware circuit may be an integrated circuit. Examples of a processor comprise, but are not limited to, a central processing unit (CPU), a network processor, a vector processor, a digital signal processor (DSP), a field programmable grid network (FPGA), a programmable logic assembly (PLA), an application specific integrated circuit (ASIC), a programmable logic circuit and a controller.

“Coupled”, within the meaning of the invention, means connected, directly or indirectly with one or more intermediate elements. Two elements can be in physical contact, coupled mechanically or electrically or connected by a communication channel.

“Associated”, within the meaning of the invention, means two elements coupled or having a relationship in a database.

In the remainder of the description, the same references are used to designate the same elements.

Although many solutions have been developed to allow the targeting of personalized digital media to each consumer, in particular with a view to offering products and/or services as close as possible to their needs, the result is that, very often, the consumer sees himself flooding with relatively irrelevant information, whether at dedicated advertising spaces on social networks or else by advertisements that are sent to him in his e-mail box. However, a consumer generally seeks to equip himself with products or carry out activities that will be most suited to his lifestyle, particularly if he practices a sport. This is especially true for footwear items for which an unsuitable choice can have consequences on the performance delivered by the athlete while being equipped and even cause serious damage to the feet of said athlete. Indeed, each person has a very particular way of moving or adopting a resting position.

Very often, to be able to identify a supinator or over-pronator stride, tests and analyzes of the stride by a specialist are necessary. Thus, many consumers do not know what type of stride they practice and whether it is pronounced or not. In addition, this is only one parameter among others, just as important in identifying products or services that are truly suitable for a consumer. As a result, consumers can easily choose inappropriate articles or services, such as footwear items that can cause long-term damage to their feet, such as the appearance of bony prominences or bone calcifications due to repeated friction of the shoe on the foot.

While current targeting techniques are mainly based on the analysis of Internet search data, the present invention allows the acquisition and processing of direct information to refocus the consumer at the center of the process. In particular, in order to avoid permanent discomfort, a decrease in performance as well as the appearance of physical damage to the athlete's feet, the inventors have developed a system, a computing device and a method allowing to deliver to the consumer one or more digital media really corresponding to his activity, his mobility and/or his posture, whether or not he is athletic.

Furthermore, it allows to anticipate needs based on information not expressed by the user/consumer. In particular, the invention may be based on parameters not expressed and often unknown to the user himself and include one or more of the following three classes:
the foot as an organ, where the morphological parameters of the foot will be taken into account in particular with, for example, differences between the right foot and the left foot between a righty person and a lefty person;
The functions of the foot (e.g. biomechanical parameters), where the basic movements/posture will be taken into consideration but also the more global activities such as the sports practiced; and/or
the structural and design parameters of the shoe which can be formalized in the form of a 3D file.

Thus, the present invention can allow to select personalized digital media for a user which can be based on the identification of a match between the foot, the activity of the foot and the shoe on the one hand and allow to follow continuously this match for example by other parameters such as comfort, fatigue, sweat, and/or performance.

According to a first aspect, the invention relates to a system 1 for distributing digital media. In particular, and as illustrated in connection with FIGS. 1 and 2, such a system is configured to distribute personalized digital media 401 according to distribution rules 322 and digital media 301.

The distribution rules 322 in the context of the invention may correspond to data allowing to determine the methodology for distributing digital media, for example according to mobility or posture parameters and/or shoe parameters.

In particular, a distribution repository 311 advantageously comprises distribution rules 322 for digital media 301. Indeed, in order to best personalize the supply of digital media 301 to a consumer, the distribution rules 322 can consist of a series of instructions, encoded in the distribution repository 311, which define the conditions for sending a digital media 301 to a presentation computing device 20.

The distribution rules 322 can be predefined and/or contextual. The distribution rules 322 and more generally the distribution repository 311 could be in the form of contextual dispatchers. A contextual dispatcher can take into account, for example, activity, age, and other information relating to users or their behavior to select the appropriate digital media. Furthermore, the distribution rules 322 and more generally the distribution repository 311 may take social media and relationship networks into consideration.

According to one embodiment, the distribution rules 322 and more broadly the distribution repository 311 can integrate a prediction model configured to refine the recommendations according to the values that led to the selection of personalized media and to the actions taken after the transmission of personalized media. Learning can be supervised or unsupervised learning. Thus, advantageously, the distribution system 1 according to the invention is configured to train and implement one or more algorithms. These algorithms may have been built from different learning models, in particular supervised or unsupervised partitioning. The algorithm can be derived from the use of a supervised statistical learning model selected for example from kernel methods (e.g. Support Vector Machines SVM, Kernel Ridge Regression) described for example in Burges, 1998 (Data Mining and Knowledge Discovery. A Tutorial on Support Vector Machines for Pattern Recognition), ensemble methods (e.g. decision trees) described for example in Brieman, 2001 (Machine Learning. Random Forests), k-means partitioning, decision trees, logical regression or neural networks described for example in Rosenblatt, 1958 (The perceptron: a probabilistic model for information storage and organization in the brain) or else deep learning (Kernel Methods for Pattern Analysis Hardcover—Illustrated, Cambridge University Press, 2004; Machine learning techniques on ultra-low energy microcontrollers: TinyML, Machine Learning with TensorFlow Lite on Arduino and Ultra-Low-Power Microcontrollers, O'Reilly, 2020; Dimensionality reduction techniques for hyper-dimensional data, Topological Methods in Data Analysis and Visualization V: Theory, Algorithms, and Applications Mathematics and Visualization, Springer Verlag, 2020.).

Preferably, the distribution rules 322 can take into consideration the shoe parameter values 201. By way of non-limiting example, the distribution repository 311 can comprise distribution rules 322 taking into account a composition of the sole, a category of the footwear item, performance values of the footwear item, aesthetic parameter values of the footwear item.

Preferably, the distribution rules 322 can take into consideration the mobility or posture parameter values 101 measured by the connected sole 10. By way of non-limiting example, the distribution repository 311 can comprise distribution rules 322 taking into account the most frequent type of activity (e.g. the type of sport) as well as the mobility or posture characteristics of the user such as: pronation/supination values, impact force values, pace length values, contact time values, acceleration values, angular speed values, sole orientation values, propulsion speed, fatigue rate, Fick angle, a propulsion direction and a deceleration direction.

Thus, the distribution rules 322 advantageously allow to condition the selection and the transmission of digital media 301, for example according to the shoe parameter values 201 and the mobility or posture parameter values 101 measured by the connected sole 10.

The digital media 301 which will be personalized within the framework of the present invention can be in any form allowing a transfer of information to the user. For example, a digital media 301 encodes one or more information in a format selected from the following formats: Text, images, video, sounds and their combinations.

In the context of the invention, the personalized digital media 401 are distributed to presentation computing devices 20 each coupled to at least one connected sole 10 coupled to a footwear item.

In particular, a personalized digital media 401 could correspond to a digital media selected from a list of digital media 301. A personalized digital media 401 could also correspond to a digital media 301 modified so as to integrate data from the mobility or posture parameter values. This can for example allow to personalize information or data according to the mobility of the consumer.

Similarly, a personalized digital media 401 could also correspond to a digital media 301 modified so as to integrate data resulting from shoe parameter values. Thus, this allows to personalize information or data according to characteristics of the consumer's footwear item.

Preferably, a digital media is modified so as to integrate data derived from shoe parameter values and data derived from mobility or posture parameter values. Even more preferably, a digital media is modified so as to integrate data derived from shoe parameter values, data from mobility or posture parameter values and personal data on the user of the connected sole(s) 10.

A footwear item 11 in the context of the invention may for example correspond to a shoe such as a city shoe, a slipper, a sports shoe, a flip-flop or else a sandal.

As will be described, provision is made for an identifier, associated with a connected sole 10, to be able to be transmitted together with the mobility or posture parameter values 101 to precisely identify the footwear item 11, or else that said footwear item 11 is directly identified by the consumer when entering the latter on a suitable interface of the presentation computing device 20.

A footwear item 11 may be associated with shoe parameters. Shoe parameters aim at describing the characteristics of the footwear item used during the operation of the connected sole.

These shoe parameters may concern both the technical characteristics of the shoes and the aesthetic or behavioral characteristics. This may include the shape, the properties of the materials used, optionally the mechanical characteristics, in particular of the sole.

For example, the shoe parameters may include technical parameters such as, by way of non-limiting examples:
a value associated with the cushioning,
a value associated with the “drop” of the sole of the footwear item 11, that is to say the difference in height between the heel and the front of the foot or in other words the difference between the thickness of the sole at the back and at the front of the footwear item 11, a high “drop” resulting in increased use of the heel in the attack phase of the foot during a stride and therefore puts more strain on the athlete's knees,
a value associated with flexibility,
a value associated with robustness,
a value associated with the presence of a reinforcement, and/or
a category value describing in particular whether the footwear item 11 belongs to a stride of the universal type, an over-pronator or a supinator stride.

Shoe parameters may also concern:
the type of sport to which the footwear item 11 corresponds,
the brand associated with the footwear item 11, and/or
an identifier of the connected sole 10 associated with the footwear item 11.

Furthermore, the shoe parameter values 201 can include structural parameter values indicating the different elements constituting a predetermined model of footwear item 11. The shoe parameter values 201 can thus indicate the materials used, the type of shoe (high, low, hiking, for work) the presence of a stiffener, a hard toe, a sole, a shank, a mounting insole, a sock liner, a stem. More particularly, the shoe parameter values 201 can describe the shape of each of the elements constituting the footwear item 11 and their arrangement.

The shoe parameter values 201 may further include structural or geometric parameter values. The geometric parameter values can indicate the dimensions relating to each of the structural elements of the footwear item 11.

Finally, the shoe parameter values 201 can also include aesthetic parameter values indicating for example for each structural element a color, and/or a particular aesthetic pattern.

The shoe parameters 201 of a digital media distribution system 1 according to the invention may further include reference use parameters. The reference use parameters may, for example, correspond to activity types, activity frequency or else activity intensities. It will be possible, for example, for a shoe to be associated with a walking activity, with a high weekly frequency and with certain values of impact force and/or propulsion force.

In order to facilitate the identification of the connected sole and by extension that of the footwear item 11 equipped with said connected sole 10, the connected sole 10 can comprise an integrated circuit 12, more commonly called an electronic chip comprising a unique identification number. The shoe parameters 201 can thus include the unique identification number of the integrated circuit 12 present in the connected sole 10 of the footwear item 11.

Advantageously, the footwear item 11 can also include an embedded code, preferably electronically embedded, for example at the connected sole 10 and thus allowing a two-factor authentication of the footwear item 11.

The footwear items 11 will be equipped with one, preferably two connected soles 10 (that is to say one connected sole 10 per footwear item).

A connected sole 10 is configured to generate raw data from which it is possible to calculate mobility or posture parameter values 101.

The parameters called “dynamic” parameters relating to the use of the footwear item 11, such as mobility or posture parameters 101 can correspond to biomechanical parameters. Thus, the mobility or posture parameters 101 can be selected, for example, from: pronation/supination values, impact force values, pace length values, contact time values, acceleration, angular speed values, sole orientation values, propulsion speed, fatigue rate, Fick angle, a propulsion direction and a deceleration direction. Such parameters may also correspond to the pace length, the contact time, time of flight, lameness, propulsion force, balance and several other parameters relating to the user and describing his gait, postures and his movements. Preferably, the mobility or posture parameters 101 may include the number of paces, pronation/supination data, force or propulsion speed, impact force or speed, stride length, orientation in flight of the foot, fatigue. Furthermore, the mobility or posture parameters 101 may also include the analysis of the user's main activities (walking, running, basketball, . . . ).

Advantageously, the most relevant mobility or posture parameters 101 in the context of the present invention are the parameters related to the health of the person such as the impact force parameter on contact with the ground (in particular if it shows a high value), pronation and/or supination parameters (in particular if they show a high value), and/or lameness. Furthermore, the wear parameter of the footwear item is also very relevant, in particular because excessive wear of the footwear item can cause injuries.

Even more preferably, the mobility or posture parameters 101 include at least: the impact force parameter, the pronation parameter and/or the supination parameter.

Furthermore, some parameters may be of little use alone but prove to be very interesting combined with other parameters obtained from the user or from surrounding parameters such as temperature, humidity, indoor or outdoor activity. Thus, a solution according to the invention can take into account the values of personal parameters of the user such as his weight, his height, his age, his possible pathologies, nutritional diet and/or his sleep time.

Furthermore, a connected sole 10 can be configured to generate raw data from which it would be possible to calculate a wear parameter value of the shoe.

The data relating to the mobility or posture parameter values 101 can be stored on a dedicated server then downloaded and/or viewed by the user on the presentation computing device 20.

A shoe wear parameter can advantageously correspond to a period before which it would be desirable to change shoes. Alternatively, it can correspond to a percentage or any other value that can provide an indication of the state of wear, which is preferably structural, of the shoe. Indeed, a user can know the state of aesthetic wear of a footwear item, however, it remains difficult to determine the damage undergone by the structures guaranteeing cushioning or adequate hold for the user and his activity. However, unconventional uses can lead to premature wear of the shoes.

A shoe wear parameter value may be calculated from data generated by a connected sole 10. In particular, a shoe wear parameter value may be calculated by a connected sole, a presentation computing device 20 or else a calculation computing device 30.

A wear parameter value can for example be calculated from a number of paces taken with this connected sole. Furthermore, the number of paces taken may preferably be weighted by one or more mobility or posture parameter values 101 such as a speed (that is to say median or average speed), an acceleration (that is to say median or average acceleration), a value of supination or high pronation or a particular gait pattern. Thus, a wear parameter value will preferably be calculated from one or more mobility or posture parameter values 101, more preferably coupled to shoe parameter values.

Even more preferably, a wear parameter value will preferably be calculated from a comparison of the values of one or more mobility or posture parameters 101 obtained over time. For example, a decrease over time in accelerations, in particular vertical accelerations, and/or propulsion speeds can be considered as a wear marker.

Advantageously, the shoe parameter values 201 or the mobility or posture parameter values 101 can further include a wear datum for the footwear item 11 associated with a connected sole 10. In particular, the wear datum of the footwear item 11 can correspond to a wear parameter value of the shoe (that is to say of the footwear item 11). The wear datum of the footwear item allows to describe a type of wear of the footwear item 11 and can be regularly updated according, for example, to mobility or posture parameters 101. Still with a view to personalized targeting, sending a personalized digital media 401 may be subject to the wear datum, that is to say on the condition that the value of the wear parameter exceeds a predetermined threshold value, implying that the footwear item 11 of the consumer must be replaced.

A connected sole 10 is associated, preferably coupled directly or indirectly, to a presentation computing device 20. Preferably, the connected sole and the presentation computing device 20 share a unique identifier associated with the at least one connected sole 10. Thus a presentation computing device 20 can be coupled to several independent connected soles 10 or to several pairs of connected soles 10.

To perform the measurements and the acquisition of the raw data allowing to determine mobility or posture parameter values of the consumer, the connected sole 10 preferably comprises a device integrated in an electronic unit inserted into at least one of the two inner and/or outer soles of shoes of the same pair.

This device integrated into an electronic unit can generate raw data on the consumer's posture, gait and/or activity. These raw data can be processed in each of the units, in one of the units or else in a computing device to which the raw data will have been sent.

Each of the electronic units can be configured to communicate to the unit of the other sole and/or to a presentation computing device 20 external to said connected sole 10, raw data or processed data that may correspond to the mobility or posture parameter values 101 in particular by short wave or high frequency signals of the Bluetooth type.

The electronic unit may in particular comprise a PCBA (for “Printed circuit board assembly”) type electronic card and a power source, said unit advantageously having an internal data storage memory, allowing the processing and exchange of measured data.

Preferably, the electronic unit comprised in the connected sole 10 includes an inertial platform configured to generate a set of data on the mobility or posture of an athlete, or more generally of a user, of said connected sole 10.

While a user is walking, the inertial platform acquires signals representative of a movement parameter (acceleration and/or speed, for example angular speed) of the foot along the axes X, Y, Z. Furthermore, these data can then be processed to generate at least one acceleration signal. The inertial platform is for example made up of at least one accelerometer and one gyroscope. Preferably, it includes several accelerometers and gyroscopes.

The electronic unit can also include one or more magnetometers so as to acquire three additional raw signals corresponding to the magnetic field values in three dimensions.

The electronic unit may moreover include other sensors, in particular an inclinometer, a barometer, an accelerometer-type motion sensor, a temperature sensor and an altimeter to benefit from increased precision. Furthermore, the electronic unit can be coupled to other sensors, for example distributed in the sole, such as pressure sensors or force sensors. In particular, the pressure and/or force sensors can include electrodes and be made of piezoelectric materials.

Preferably, the electronic unit of the connected sole 10 includes a data processing module configured to transform all the data generated using predefined algorithms. Indeed, in a system 1 according to the invention, the electronic unit of the connected sole 10 can advantageously be able to process the data received via the sensors located in the inner and/or outer soles according to one or more algorithms.

The processing module can be used to generate biomechanical gait parameter values or else mobility or posture parameter values. Advantageously, the data processing module is capable of transforming all the data, that is to say the raw data measured by the sensors of the connected sole 10, into at least one biomechanical parameter of the gait or mobility or posture parameter values, said biomechanical gait parameter preferably being selected from: posture, type of stride, namely universal stride, over-pronation, supination, impact force, impact zone, the pace length, the contact time, the flight time, the lameness, the propulsion force, the balance and several other parameters relating to the user and describing his gait, his postures and his movements such as in particular the number of paces, the number of daily paces, the number of weekly paces, the variance of the number of paces over a given period.

By way of non-limiting example, a unit of a connected sole 10 is configured in a “Slave” unit, which receives the data from the connected sole 10 and therefore from the corresponding shoe and transmits them to the “Master” unit, or main unit. The main unit receives the data from the Slave unit, processes it by comparing it to its own data and generates information on the posture of the user in general and his feet in particular.

Thus, an electronic unit can transmit to the presentation computing device 20, in real time or offline, raw data on the mobility or posture of the consumer, pre-processed data that can be used to calculate mobility or posture parameter values 101 or else directly the mobility or posture parameter values 101.

Advantageously, the transformation by the data processing module of the electronic unit of the connected sole 10 can advantageously comprise the segmentation of the data into a plurality of phases. Preferably, said data processing module is able to segment a pace into at least four phases such as: the impact phase (corresponds to the precise moment of contact of the foot with the ground), the bearing phase (takes place from the impact phase until the heel lifts off the ground), the propulsion phase (begins when the heel leaves the ground and ends when the first toe leaves the ground) and the flight phase (starts when the first toe leaves the ground and ends when the heel touches the ground).

More particularly, the division or segmentation of the pace can allow to identify the main bearing areas and/or the main bearing moments for the user. Thus, the connected sole 10 can be used to measure the shape of the pace during walking or any other activity of the user in order to determine information related to the posture of the user.

The information generated can then be transmitted to the second unit of the other connected sole 10 present in the corresponding second shoe in the case of a “master-slave” configuration of the electronic units, or directly to the presentation computing device 20, by an emission of signals which may be of the Bluetooth type. Preferably, the connected sole 10 advantageously comprises communication means suitable for sending Bluetooth type signals. Preferably, the connected sole 10 comprises communication means configured to connect to the communication network R1 and to emit signals to the calculation computing device 30.

Advantageously but in a non-limiting manner, the electronic unit of a connected sole 10 can include a data memory, configured to memorize all or part of the transformed data and/or the data generated by the processing module of said electronic unit. Furthermore, the electronic unit of the connected sole 10 may include communication means. Thus, in particular, each of the units, whether Slave or Master, is designed so as to be able to communicate independently with the other and/or directly with the presentation computing device 20 in order to be able to exchange its own information on the posture/the movement/the activity of the shoe in which the connected sole 10 is inserted and therefore by extension of the athlete's foot, from which it has received the data.

Preferably, the transmission of the data measured by at least one connected sole by the communication means is operated via a wireless protocol such as wifi, 3G, 4G, and/or Bluetooth. Preferably the communication protocol is a BLE or ANT+ protocol. These communication protocols allow low power consumption.

Furthermore, the electronic unit of the connected sole 10 includes an energy source. The energy source is preferably of the battery type, which is rechargeable or not. Furthermore, it can be associated with a system for charging by movement or by external energy. The system for charging by external energy can in particular be a system for charging by wired connection, a system for charging by induction or else a photovoltaic system.

Finally, the connected sole 10 includes a processor. Such a processor is associated, that is to say includes, or cooperates via an electrical bus with, a data memory responsible for recording one or more computer programs. Such a computer program consists of a set of program instructions interpretable or executable by the processor and allowing to control the functions associated with the sensors as well as a processing of the measurements carried out by said sensors.

As described in FIG. 1, a system 1 in accordance with the invention comprises an associated presentation computing device 20, preferably coupled to at least one connected sole 10.

Such a presentation computing device 20 comprises a processing unit, for example in the form of a microcontroller, cooperating with a data memory, possibly a program memory, said memories possibly being dissociated. The processing unit cooperates with said memories by means of internal communication buses. A presentation computing device 20 may further include first communication means cooperating with the processing unit and providing wireless proximity communication with the connected sole 10 within communication range, in particular via the Bluetooth® communication protocol. It may also include second communication means of the “long distance” type also cooperating with the processing unit. These second communication means allow such a presentation computing device 20 to be able to transmit to a remote entity, for example a computing server, data, such as the posture and mobility parameter values 101 through messages distributed by a network R1 using, for example, GSM, GPRS or satellite technologies. To operate, that is to say so that the processing unit can execute program instructions recorded in the data memory, the presentation computing device 20 includes an electrical energy source, in the form of one or several batteries for example.

A presentation computing device 20 in accordance with the invention consists advantageously but in a non-limiting manner of a smartphone configured to receive one or more digital media 301 and possibly mobility or posture parameter values 101 generated by the connected sole 10. The presentation computing device 20 is further capable of retransmitting the mobility or posture parameter values 101 to another computing device such as a computing server. For this purpose, the invention provides that an appropriate application can be designed to be installed in a memory of said presentation computing device 20 to allow its user to consult the mobility or posture parameter values 101 or else digital media 301. Such an application could, in addition, be installed in any other computing device, possibly via some software adaptations, thus a presentation computing device 20 according to the invention can, for example, consist of a touch pad, a personal computer, a connected watch or more generally any connected object comprising suitable hardware and software resources.

As described previously, a presentation computing device 20 is preferably configured to receive data transmitted by at least one connected sole 10. These data may include one or more parameter values associated with the gait of a consumer using the footwear item 11. In general, the raw data, the pre-processed data and/or the mobility or posture parameter values 101 generated by the connected sole 10 can be transmitted to the presentation computing device 20.

Preferably, the mobility or posture parameter values 101 are transmitted via a dedicated communication network between the presentation computing device 20 and the connected sole 10, such as for example according to a Bluetooth® communication protocol. Advantageously, but in a non-limiting manner, each connected sole 10 or presentation computing device 20 can encode, in a dedicated data memory, an identifier relating to the model of the footwear item 11 in which said connected sole 10 is inserted. In a particular embodiment, the user (that is to say consumer) or the athlete, can enter directly via a suitable interface of the presentation computing device 20, the model of the footwear item 11 comprising the connected sole 10.

Once the mobility or posture parameter values 101 have been received by the presentation computing device 20, said mobility or posture parameter values 101 are sent, through the communication network R1, to a calculation computing device 30 taking the form here of a computing server. Alternatively, a connected sole 10 can be configured to send the mobility or posture parameter values 101 directly to the calculation computing device 30 without going through the presentation computing device 20. Thus, a system 1 in accordance with the invention comprises one or several calculation computing devices 30 configured in particular to load parameter values. The loading of the parameter values can in particular correspond to the loading into memory of these data. A calculation computing device 30 may be configured to obtain these values and in certain cases it may be configured to calculate them for example from raw data.

Alternatively, raw data or pre-processed data can be sent by the at least one connected sole 10 to the presentation computing device 20 or to a computing server 30. When the presentation computing device 20 receives raw data or pre-processed data it is advantageously configured to calculate mobility or posture parameter values 101 from these data and to transmit these mobility or posture parameter values 101 to the computing server 30 or it can directly transmit these raw or pre-processed data to the computing server 30. The computing server 30 will then be configured to calculate mobility or posture parameter values 101 from these raw or pre-processed data.

Furthermore, in an embodiment not shown, the calculation computing device 30 corresponds to the electronic unit of the connected sole and it is not necessary for the connected sole 10 to transmit raw data, preprocessed data or mobility or posture parameter values 101.

Thus, the calculation computing device 30 has on the one hand shoe parameter values 201 associated with the footwear item 11 worn by the consumer as well as mobility or posture parameter values 101 calculated from raw data generated by the connected sole 10 comprised in said footwear item 11.

A calculation computing device 30 in accordance with the invention includes a processing unit, a data memory, communication means allowing said calculation computing device 30 to cooperate with other computing devices via a long-range communication network R1 of the Internet, LoRa or Sigfox type, or any other equivalent communication network. Such a calculation computing device 30 can further comprise a data memory for storing any computer program whose program instructions, interpretable or executable by the processing unit, allow to automatically adapt the operation of a conventional server so that it becomes a computing server 30 in accordance with the invention. The various electronic components of such a calculation computing device 30 cooperate with the processing unit by wired communication bus.

The data memory can be partially or entirely electrically erasable in order to be updated. Generally, a section of said data memory is not erasable by construction, or is protected against such erasure by a security mechanism. Such a memory section records in a durable manner, in particular the value of a unique identification datum characterizing a connected sole 10 with regard to other pair connected soles.

Depending on the arrangements, such a calculation computing device 30 can also integrate any hardware and/or software component, such as in particular a hypervisor, allowing to view all or part of the hardware and/or software components of said calculation computing device 30.

For example, a calculation computing device 30 may correspond to a computing server accessible remotely, a computer, a connected object such as a telephone or a tablet or it may correspond to the electronic unit of a connected sole.

Advantageously, the calculation computing device 30 is configured, in particular through processors and data memories, to load mobility or posture parameter values 101 calculated from raw data generated by at least one connected sole 10; load shoe parameter values 201; Load digital media 301 and a distribution repository 311 including digital media 301 distribution rules 322; transmit a personalized digital media 401 to at least one of the presentation computing devices 20 coupled to the at least one connected sole 10 having generated said raw data.

As will be detailed below, in the context of the present invention, the calculation computing device 30 is preferably configured to receive mobility or posture parameter values 101 calculated from raw data generated by at least one connected sole 10. These mobility or posture parameter values 101 are also associated with one or more presentation computing devices 20 such as for example a smartphone, a tablet or a connected watch. This or these presentation computing devices 20 are preferably mobile and are each associated with at least one connected sole 10.

The calculation computing device 30 could for example receive an identifier associated with a presentation computing device 20 associated with a connected sole 10 coupled to a footwear item 11. Thus, it could preferably be configured to establish a relationship graph between an identifier of a given connected sole equipping a particular footwear item also associated with a unique identifier, one and/or the other of these unique identifiers being listed in a presentation computing device 20. These data may or may not be associated to a unique identifier of a user.

The calculation computing device 30 can also be configured to calculate mobility or posture parameter values 101 calculated from raw data generated by at least one connected sole 10 or from pre-processed data themselves generated by at least one connected sole 10.

Furthermore, the calculation computing device 30 is preferably configured to load shoe parameter values 201. These shoe parameter values each relate to a footwear item 11 coupled to the at least one connected sole 10. For example, the calculation computing device 30 will be able to load a weight value of the footwear item 11 coupled to the connected sole 10 having generated the raw data at the origin of the mobility or posture parameter values. These shoe parameter values may for example come from a local or remote data memory. Preferably the shoe parameter values may be stored on a media computing device 40 which will be described later.

Advantageously, the calculation computing device 30 can be configured to further load environmental parameter values 701, said environmental parameter values 701 corresponding to environmental conditions measured during use of the footwear item 11 associated with the at least one connected sole 10. Such environmental parameters 701 can be measured by one or more dedicated sensors positioned in the connected sole 10 or downloaded directly from the web from the geographical position of the connected sole 10.

Furthermore, the system or the method according to the invention can allow or include the detection of the position of the user and indicate to him according to the establishments in his vicinity a personalized digital media. Such personalized digital media could correspond to an offer that would be adapted to his profile in one of the nearby establishments or for use in one of the nearby establishments.

The environmental parameters can be determined from the geographical coordinates of the connected sole 10 then classified according to a type of use environment. By way of non-limiting examples, the environmental parameters may correspond to a geographical area, a country, an urban area, a rural area, an average temperature, an average humidity rate, a level of precipitation, a rate of sunshine during the use of the footwear item 11 equipped with the connected sole 10, with for example reference environmental parameters for the footwear item 11 indicating a type of recommended use environment.

The environmental parameters may further correspond to a “virtual” type of use, particularly in connection with the field of video games, more particularly simulation video games.

The calculation computing device 30 may further be configured to identify a triggering event and to transmit 540 the personalized digital media 401 to the at least one presentation computing device 20 when a triggering event has been identified.

“Triggering event” means information received or calculated, for example, by the connected sole 10, by the calculation computing device 30 or by the presentation device 20 in connection with the shoe parameters 201, the mobility or posture parameters 101, the environmental parameters 701 or conventional or unconventional use and/or the user parameters 801.

In order to improve the degree of personalization of a personalized digital media 401, the presentation device 20 or else the connected sole 10 can occasionally emit a presentation message indicating its geographical position, to the calculation computing device 30. Depending on the geographical position, the calculation computing device 30 may indicate the presence of points of sale of footwear items within a radius of a predetermined distance and in particular that a footwear item offer would be adapted to the profile of the user of the footwear item 11 equipped with the connected sole 10 in one of the identified points of sale.

By way of illustrative example, the personalized digital media 401 can be sent according to an event related for example to a deterioration in the condition of the footwear item, more particularly to a wear datum or else to a bad use of the footwear item associated with the connected sole 10. In this specific case, the aim sought is to provide highly personalized digital media 401 which is really dependent on the needs of the user at a given moment.

In order to improve the degree of personalization of a personalized digital media 401, the calculation computing device 30 is configured to further load user parameter values 801, said user parameter values 801 corresponding to morphological data of the user of the footwear item 11 associated with at least one connected sole 10. These morphological data may correspond to morphological data of the user's feet such as, for example, the dimensions of the foot (length/height of the arch, length/width of the foot), size and position of the toes, and/or shape of the arch of the foot (e.g. sunken or exaggerated for respectively flat foot or hollow foot).

The user parameter values 801 may in particular correspond to the values of personal parameters mentioned above and may also include the type of sport practiced by the user of the connected sole 10, the level at which the latter practices said sport (leisure, amateur, professional). Furthermore, the calculation computing device 30 is preferably configured to load digital media 301 and a distribution repository 311 including digital media 301 distribution rules 322. These digital media 301 and this distribution repository 311 could for example come from a local or remote data store. Preferably the digital media 301 and the distribution repository 311 can be stored on a media computing device 40 which will be described later.

Furthermore, the calculation computing device 30 is preferably configured to transmit personalized digital media 401 to at least one of the presentation computing devices 20 associated with the at least one connected sole 10 having generated said raw data from which the mobility or posture parameter values 101 have been generated. The personalized digital media 401 transmitted can for example be selected, within the digital media 301 received, according to the distribution rules 322, the mobility or posture parameter values 101 and the shoe parameter values 201 of the footwear item 11 coupled to at least one connected sole 10.

This allows the reception by the presentation computing device 20 of truly personalized and in particular personalized digital media on the basis of the physical activity of the consumer, preferably of mobility or posture, and the characteristics of the footwear items that he used. Thus the personalization reaches a higher level compared to the existing one by being based on the social habits (e.g. purchase) of the consumer and on these physical habits.

Furthermore, the calculation computing device 30 may be configured to identify unconventional uses of footwear items 11.

Indeed, as mentioned, the shoe parameters can include reference use parameters. Therefore, shoe parameter values may include reference use parameter values. In this context, the calculation computing device 30 can be configured to calculate use parameter values from the mobility or posture parameter values 101 then to compare these use parameter values calculated with the reference use parameter values.

Within the framework of the generation of recommendations, such an identification may include an operation of comparing the reference use parameters with use parameters calculated from the mobility or posture parameters 101. In particular, the labeling of a shoe parameter in connection with the type of stride associated with the corresponding footwear item 11 can constitute a quite relevant reference use parameter. In this case, this allows to calculate use parameters from the mobility or posture parameter values 101, such as the type of stride, and to identify a use of footwear items 11 adapted for a stride of the supinator type in an athlete with a stride of the over-pronator or universal type and therefore using unsuitable footwear items 11.

One or more of the mobility or posture parameters can for example be labeled as reference use parameters. For example, if it turns out that said mobility or posture parameter values calculated from raw data measured by a connected sole 10 are respectively greater than a predetermined threshold, then unconventional use can be identified and a message alert generated. Such identification may include an operation of comparing said mobility or posture parameter values with the reference use parameters. In this case, this allows to calculate use parameters from the mobility or posture parameter values 101, such as the impact force and/or the propulsion force, and to identify a use of footwear items 11 which is adapted or not. Indeed, some footwear items 11 may have a thin sole and therefore less cushioning for a consumer having a too great impact and/or propulsion force. This inappropriate use can ultimately cause premature wear of the footwear item or, worse, damage to the knee joints, in particular due to poor shock absorption by the footwear items 11.

In order to identify an unconventional or inappropriate use of a footwear item 11 comprising a connected sole 10, the calculation computing device 30 of a digital media distribution system 1 according to the invention can be configured to receive geographical positioning data from the connected sole 10. In this case, the connected sole 10 comprises a satellite geolocation module configured to determine a position of the connected sole 10 resulting in data relating to the longitude, the latitude and possibly the altitude of said connected sole 10.

In a particular embodiment, the positioning data can be communicated to the calculation computing device 30 by the presentation computing device 20 coupled to the connected sole 10. In this case, it is the presentation computing device 20 which comprises the satellite geolocation module.

Thus, it is possible to identify an unconventional or unsuitable use by identifying a trend of use of a footwear item 11 comprising a connected sole 10 correlated with geographical parameters. This also allows to identify geographical areas with their characteristics (e.g. forest, road, track) and thus possibly to suggest new recommendations, as described above, to the consumer by offering him footwear items 11 more suited to the racing areas travelled.

As shown in FIG. 1, a system 1 in accordance with the invention may further comprise a media computing device 40.

The media computing device 40 can advantageously correspond to a server comprising a data memory configured to store a plurality of digital media 301 and a distribution repository 311.

The calculation computing device 30 may further be configured to transmit, to the media computing device 40, a confirmation message of the transmission of the digital media to the presentation computing device 20. This allows to confirm the distribution information and the media computing device will have the capacity to store information on the digital media distributed without having to know the mobility or posture parameter values. In particular, this transmission confirmation message may include an identifier associated with a presentation computing device 20, which is preferably mobile, and an identifier of the transmitted digital media.

For each transmission confirmation message received by the media computing device 40, a virtual token can be assigned to the consumer, more particularly the virtual token can be associated with the identifier of the digital media transmitted and transferred to the consumer in particular by associating the identifier of the consumer's presentation computing device 20 therewith. The virtual token can then be made available to the consumer through a dedicated application through which the consumer can purchase/subscribe to a product or service.

According to a particular embodiment, a calculation computing device 30 according to the invention can also be configured to calculate, for each connected sole 10, one or more use parameter values from the mobility or posture parameter values 101. For this purpose, the calculation computing device 30 advantageously comprises, in a dedicated data memory, a use repository. The use repository can include reference posture or mobility values with which a predetermined type of use is associated. By way of non-limiting examples, the determination of a use can be associated with a specific pattern of reference posture or mobility values. The determination of a use may also be subject to whether or not a predetermined threshold is exceeded. The person skilled in the art will appreciate that the possibilities for determining a use according to the posture or mobility parameters are very numerous and that it will be possible to configure the calculation computing device 30 to calculate one or more use parameter values according to mobility or posture parameter values 101 deemed relevant for a given use.

By way of non-limiting examples, a use parameter value may indicate a sporting, urban, recreational use of a footwear item 11. For this purpose, the calculation computing device 30 can be configured to identify in the mobility or posture parameter values 101 previously loaded, values describing an impact force, a pace length, an acceleration, a speed of propulsion and a time of flight. In particular, the calculation computing device 30 may be configured to compare values describing an impact force, a pace length, an acceleration, a propulsion speed, and a time of flight to a plurality of reference value patterns for these posture and mobility parameters taken alone or in combination. The person skilled in the art will understand that in the context of sports practice one or more parameters may be taken into account to identify a use of a footwear item. In particular, a sports practice of the running type could, for example, be characterized by values describing an acceleration, a particular time of flight or else a pace length or a running speed or else a combination of these parameters. It may also be interesting to identify which footwear item 11 is used for a given use by a user category. For this purpose, the mobility or posture parameter values 101 taken into account can include pronation and/or supination values. In the context of problems more related to the field of health, affecting for example the gait of a user, other mobility or posture parameter values could be taken into account to identify which footwear item 11 is used, for a given use, by a user category. In particular, the posture or mobility parameters, affecting the user's gait, which can be analyzed are the presence of lameness, a fatigue rate, orientation values of the sole, balance. Thus, it will be possible to determine on the one hand a particular use of a footwear item in the context of a sports practice for example, for a given user category.

The calculation computing device 30 according to the invention is further configured to determine, for each connected sole 10, an association index value between the use parameter values 101 and the shoe parameter values 201. This index value can in particular allow to determine whether the footwear items are used as planned during their design and in connection with their mechanical properties. Unanticipated uses may correspond to trends on the one hand but also to risks on the other hand. The association index value can be considered as the result of a correspondence analysis between the use parameter values 101 and the shoe parameter values 201. The system according to the invention could therefore in certain aspects be considered as a correspondence engine or even a recommendation system which establishes correspondence between activities and the initial destination of the footwear items, knowing that it can advantageously integrate many other parameters such as the terrain, the environment, the geography, the weather, . . . .

For this purpose, the calculation computing device 30 advantageously comprises, in a dedicated data memory, an association repository. The association repository includes for shoe parameter values 201, a reference use parameter value. By way of non-limiting example, it is provided that each footwear item 11 can be described through shoe parameter values 201. One or more of the shoe parameter values 201 can advantageously be associated with a use parameter value. The person skilled in the art will appreciate that depending on the recommended use, for a sporting activity for example, a footwear item has shoe parameter values 201 specific to said recommended use. Thus, the calculation computing device 30 is advantageously configured to determine an association index value which can, for example, be in the form of a value comprised between 0 and 1 characterizing a concordance between the use value(s) of the use parameter 101 calculated from the mobility or posture parameter values 101 for the footwear item 11 whose shoe parameter values 201 have been loaded.

Indeed, it is possible that the user uses a footwear item 11 equipped with a connected sole in accordance with the invention for a rather sporting use or then for use in everyday life and therefore rather associated with a less dynamic gait. Thus, the calculation computing device 30 will be able to determine, for a series of mobility or posture parameter values 101 calculated from raw data generated by at least one connected sole 10 over a given time interval, a use parameter value. By way of non-limiting example, an association index value close to 1 will mean that the calculated use parameter value(s) correspond to the reference use value associated with the shoe parameter values 201 comprised in the association repository for the footwear item 11. On the contrary, an association index value close to 0 will mean that the calculated use parameter value(s) do not correspond to the reference use value associated with the shoe parameter values 201 comprised in the association repository for the footwear item 11.

The calculation computing device 30 in accordance with the invention can further be configured to identify a conventional and/or unconventional use, by a user, of a footwear item 11 including a connected sole 10. Such an identification can correspond to a comparison operation of the use parameter value(s) 101 calculated with a reference use value comprised in the association repository for the footwear item 11 comprising the connected sole 10 whose generated data allowed to calculate the use parameter value(s). In the case where the reference use value is equal to the calculated use parameter value, then the use of the footwear item 11 including the connected sole 10 is considered as conventional. Conversely, if the reference use value is not equal to the calculated use parameter value, then the use of the footwear item 11 including the connected sole 10 is considered to be unconventional.

In order to allow the manufacture of a footwear item 11 adapted to the use of a user, the calculation computing device 30 can also be configured to generate production parameters relating to the manufacture of footwear items 11 on the basis of the determined association index values between the use parameter values and the shoe parameter values 201. Such production parameters can comprise the materials used, their shape and their dimensions, but also the design of the footwear item (for example colors, logos, applications, etc.) or its functional properties (for example impermeability, cushioning, etc.) and can then be updated according to the use parameter values 101 obtained.

As illustrated in FIG. 2, a system 1 in accordance with the invention may comprise an anonymization server 50 or pseudonymization server. Indeed, in order to limit the dissemination of personal data, a system according to the invention may include a computing device such as an anonymization server 50 or pseudonymization server configured to modify identification data or personal data possibly associated with the raw data, the pre-processed data or the mobility or posture parameter values 101. This allows information to be separated to guarantee confidentiality and long-term management of personal data. This modification may be in the form of data encryption, data anonymization or else data pseudonymization.

Thus, an anonymization server 50 can comprise a data memory configured to memorize a sensitive data identification repository and an anonymization repository. It can also be configured to memorize a repository of correspondence between sensitive data and anonymized data. The data memory may comprise transient memory and/or non-transient memory. The non-transient memory can be a medium such as a CDrom, a memory card, a local hard disk or a hard disk hosted by a remote server. Preferably, the data memory takes the form of a local hard disk.

An anonymization server 50 according to the invention advantageously comprises a processing unit including a processor for the execution of at least one message analysis and processing function. In particular, all the messages sent by the presentation computing device 20 to the calculation computing device 30 (and conversely sent by the calculation computing device 30 to the presentation computing device 20) can be redirected to the anonymization server 50. Furthermore, the processing unit may include or be coupled to other means such as combinations of processors, memory as well as codes supporting instructions or to a data memory including a plurality of memorized repositories. The processing unit can in particular be configured to analyze said messages emitted by and/or intended for the presentation computing device 20. The processing unit being in particular configured to identify, from an identification repository of sensitive data stored in a data memory of said anonymization server 50, data to be anonymized in a message emitted by the presentation computing device 20. The anonymization server 50 is thus configured to generate, from the anonymization repository, anonymized data from the data to be anonymized previously identified and generate, from the anonymized data and the message emitted by the presentation computing device 20, an anonymized message. Here, data anonymization may correspond to pseudonymization. Furthermore, the anonymization server 50 may be positioned between the calculation computing device 30 and the media computing device 40.

An anonymization server 50 according to the invention further comprises communication means configured to exchange data with third-party devices, in particular with a calculation computing device 30, a presentation computing device 20 or else with a media computing device 40. The communication means can correspond to any element or combination of elements allowing to receive or send messages, via or by a communication network (for example LAN, WLAN, or PAN, log of a database, e-mail), to third-party devices. Advantageously, the communication means are configured to receive a message emitted by the calculation computing device 30 to the presentation computing device 20 or conversely a message emitted by the presentation computing device 20 to the calculation computing device 30.

In order to facilitate the encryption of the data to be anonymized and the decryption of the anonymized data, a message emitted by the presentation computing device 20 comprises a unique identifier associated with said presentation computing device 20 or else with a connected sole 10. This unique identifier can thus be associated with a secret or a private key which will be used to encrypt or decrypt part of the data (that is to say the sensitive data identified as to be anonymized before sending or the anonymized data) contained in the message.

According to another aspect, the invention relates to a method 500 for distributing digital media 301 according to distribution rules 322.

The digital media 301 are distributed to presentation computing devices 20 each associated with at least one connected sole 10, said connected soles 10 being coupled to footwear items 11.

The method according to the invention is preferably implemented by a calculation computing device 30 such as a computing server or an electronic unit of a connected sole 10.

As illustrated in FIG. 3, the method preferably includes the following steps: loading 510 mobility or posture parameter values 101 calculated from raw data generated by at least one connected sole 10, loading 520 shoe parameter values 201; loading 530 digital media 301 and a distribution repository 311 including digital media 301 distribution rules 322; and transmitting 540 a personalized digital media 401 to at least one of the presentation computing devices 20 associated with the at least one connected sole 10 having generated said raw data from which the mobility or posture parameter values 101 have been generated.

As illustrated in FIG. 4, a method according to the invention may be based on mobility or posture parameter values 101 generated by a connected sole 10 and transmitted to a presentation computing device 20 coupled to the connected sole 10.

These mobility or posture parameter values 101 can be transmitted with identification data to an anonymization server 50 responsible for anonymizing or pseudonymizing sensitive data before transmitting them to the calculation computing device 30. As shown the raw, pre-processed or processed data can be transmitted directly to the calculation computing device 30 or else not pass through an anonymization server 50.

A method according to the invention includes a step 510 of loading the mobility or posture parameter values 101 calculated from raw data generated by at least one connected sole 10. The loading step 510 can be preceded by a step of receiving the mobility or posture parameter values 101. Alternatively, it can be preceded by a step of calculating the mobility or posture parameter values 101 from raw data generated by at least one connected sole 10.

The mobility or posture parameter values 101 are preferably associated with one or more presentation computing devices 20 each being associated with at least one connected sole 10. Thus there is a virtual link, for example in the form of a database allowing to make a link between the mobility or posture parameter values 101, the shoe parameter values, one or more connected soles, one or more footwear items and preferably a consumer.

A method according to the invention includes a step 520 of loading the shoe parameter values 201. The shoe parameter values 201 preferably each relate to a footwear item 11 coupled to the at least one connected sole 10. These shoe parameter values 201 can, as mentioned above, be stored on a media computing device 40 and transmitted on request to the calculation computing device 30.

A method according to the invention includes a step of obtaining or loading 530 digital media 301 and a distribution repository 311. The distribution repository 311 can include digital media 301 distribution rules 322. Preferably, the digital media 301 and the distribution repository 311 have been sent by a media computing device 40. Alternatively they are stored on the calculation computing device 30. Indeed, the distribution rules and the contents can be sent by the media server and the media server does not have mobility or posture parameter data 101.

A method according to the invention includes a step of transmitting 540 a personalized digital media 401 to at least one of the presentation computing devices 20 associated with the at least one connected sole 10 having generated said raw data from which the mobility or posture parameter values 101 have been generated.

The personalized digital media 401 transmitted is preferably selected, within the digital media 301 received, according to the distribution rules 322, the mobility or posture parameter values 101 and the shoe parameter values 201 of the footwear item 11 coupled to at least one connected sole 10.

In order to facilitate this transmission 540, the at least one connected sole 10 may include a unique identifier and there may be a “pairing” or coupling mechanism between one or more presentation computing devices 20 and the at least one connected sole 10. A presentation computing device 20 can manage several soles or pairs of connected soles at the same time.

According to another aspect, the invention relates to a presentation computing device 20. Such a presentation computing device is preferably mobile, and it is configured to receive digital media 301 for example according to distribution rules 322.

The presentation computing device 20 according to the invention has the particularity of being associated or coupled to at least one connected sole 10.

Furthermore, the presentation computing device 20 according to the invention is advantageously configured to:
Receive mobility or posture parameter values 101 generated by the at least one connected sole 10 or raw data generated by the at least one connected sole 10;
Transmit, to a calculation computing device 30, the mobility or posture parameter values 101 generated by the at least one connected sole 10 or the raw data generated by the at least one connected sole 10;
Receive a personalized digital media 401 having been transmitted by the calculation computing device 30, said transmitted personalized digital media having been selected, within digital media 301 received by the calculation computing device 30, according to:
   a distribution repository 311 comprising distribution rules 322 sent to the calculation computing device 30 by a media computing device 40, and
   mobility or posture parameter values 101 calculated from raw data generated by at least one connected sole 10.

The present invention also relates to a method 600 for analyzing mobility or posture parameter values 101 of a user for generating a message of possible change of category of a user, said user being associated with a first category value, said method being implemented by a calculation computing device 30, including the following steps:
Loading 610 a category repository, said category repository including an association between category values and category descriptions;
Loading 620 one or more mobility or posture parameter values 101 of a user calculated from raw data generated by a connected sole 10;
Comparing 630 the mobility or posture parameter value(s) 101 of the user previously determined with a reference mobility or posture parameter value so as to determine whether a change of category is necessary;
Determining 640 a new user category value from the category repository and the first category value when the determined user mobility or posture parameter value differs from the mobility parameter value or reference posture; and
Generating 650 a message of possible change of category of a user, said message including the new value of the category of the user and the associated description.

A method according to the invention includes a data analysis taking into account the posture or mobility parameters of the users and a category change value validated by the user.

The invention further relates to a method for recommending footwear items 11 to a user, said method being implemented by a calculation computing device 30 following the reception of mobility or posture parameter values 101 generated by a connected sole 10, said method including the following steps:
receiving and analyzing a plurality of user information, originating from a plurality of users, said user information including a satisfaction index value, a posture or mobility index value associated with each of said users, and a footwear item identification datum 11;
analyzing the mobility or posture parameter values 101 generated by the connected sole 10 so as to generate a posture or mobility index value associated with the connected sole 10;
a step of comparing the posture or mobility index value associated with the connected sole 10 to the posture or mobility index values of a plurality of users; and
a step of identifying one or more footwear items 11 for which the posture or mobility index value corresponds to the posture or mobility index value associated with the connected sole 10, and for which the satisfaction index value is greater than a predetermined threshold.

A recommendation method according to the invention preferably includes a step of sending an information request message, by the calculation computing device 30 to the user, more particularly to a presentation computing device 20 from which the user is identified. The content of the message is determined at least in part by the posture or mobility index value for that user.

A recommendation method according to the invention preferably includes a continuous analysis of the mobility or posture parameter values 101 generated by a connected sole 10 of a footwear item 11 worn by the user.

A recommendation method according to the invention preferably includes an occasional analysis of the user's feeling. This analysis may in particular consist in sending a form, from the calculation computing device 30 to the presentation computing device 20 coupled to the connected sole 10 of the footwear item 11 worn by the user, comprising a plurality of questions. The user can thus answer the questions of said form from the presentation computing device 20, a response form is thus generated and sent to the calculation computing device 30. The calculation computing device 30 is furthermore configured to generate, from the question response form, a satisfaction index value associated with the footwear item 11 of the user.

Thus, such an invention can be used by a retailer who might be interested beyond the brand in establishing correspondences between a loyalty card identifier, a shoe identifier and a shoe use identifier.

In particular, the measurements of the mobility or posture parameter values of an athlete and the determination of a biomechanical parameter, within the framework of the recommendation method, can be carried out as detailed above in relation to a connected sole 10 coupled to a presentation computing device 20.

1. A digital media distribution system configured to distribute digital media according to distribution rules, wherein said digital media are distributed to at least one presentation computing device associated with at least one connected sole configured to generate raw use data, said at least one connected sole being associated with a footwear item, said digital media distribution system comprising a calculation computing device configured to:
Obtain mobility or posture parameter values calculated from the raw use data;
Obtain shoe parameter values said shoe parameter values relating to the footwear item associated with the at least one connected sole;
Obtain the digital media and a distribution repository including the digital media distribution rules; and
Transmit personalized digital media to the at least one presentation computing device associated with the at least one connected sole having generated said raw use data from which the mobility or posture parameter values have been calculated, said transmitted personalized digital media being selected, within the loaded digital media, according to the distribution rules, the mobility or posture parameter values and the shoe parameter values of the footwear item associated with the at least one connected sole.

2. The digital media distribution system according to claim 1, wherein the calculation computing device is configured to further load environmental parameter values said environmental parameter values corresponding to environmental conditions measured during use of the footwear item associated with the at least one connected sole.

3. The digital media distribution system according to claim 1, wherein the calculation computing device is configured to identify a triggering event and to transmit the personalized digital media to the at least one presentation computing device when said triggering event has been identified.

4. The digital media distribution system according to claim 1, wherein the calculation computing device is configured to further load user parameter values, said user parameter values corresponding to morphological data of a user of the footwear item associated with the at least one connected sole.

5. The digital media distribution system according to claim 1, wherein the shoe parameter values or the mobility or posture parameter values further include a wear datum of the footwear item associated with the at least one connected sole.

6. The digital media distribution system according to claim 5, wherein the calculation computing device is further configured to generate production parameters relating to manufacture of footwear items on the basis of determined association index values, shoe parameter values and the wear datum.

7. The digital media distribution system according to claim 1, wherein the calculation computing device is further configured to receive raw data generated by the at least one connected sole or pre-processed data produced from raw data generated by the at least one connected sole, said calculation computing device being further configured to calculate, from these raw data or pre-processed data, the mobility or posture parameter values.

8. The digital media distribution system according to claim 6, wherein the calculation computing device is further configured to receive the mobility or posture parameter values said mobility or posture parameter values having been generated by the at least one connected sole or the one or more at least one presentation computing devices associated with the at least one connected sole.

9. The digital media distribution system according to claim 1, wherein the at least one connected sole is directly or indirectly coupled to the at least one presentation computing device.

10. The digital media distribution system according to claim 1, wherein the mobility or posture parameter values have been calculated from raw data generated by two connected soles.

11. The digital media distribution system according to claim 1, wherein the personalized digital media includes data derived from said shoe parameter values and/or data derived from said mobility or posture parameter values.

12. The digital media distribution system according to claim 1, wherein the personalized digital media includes data derived from said shoe parameter values and/or data derived from said mobility or posture parameter values as well as personal data on a user of the connected sole(s).

13. The digital media distribution system according to claim 1, wherein the shoe parameter values include reference use parameters, the calculation computing device being further configured to identify unconventional uses, said identification including a comparison of the reference use parameters with use parameters calculated from said mobility or posture parameter values.

14. The digital media distribution system according to claim 13, wherein the calculation computing device is further configured to calculate one or more trend values based on the identification of unconventional uses correlated with geographical parameters.

15. The digital media distribution system according to claim 1, further comprising an anonymization server, said anonymization server being configured to:
identify, from a sensitive data identification repository, data to be anonymized in a message including the mobility or posture parameter values or raw data generated by the at least one connected sole or pre-processed data produced from raw data generated by the at least one connected sole;
generate, from an anonymization repository, anonymized data from previously identified data to be anonymized; and
generate an anonymized message from the anonymized data and the message.

16. The digital media distribution system according to claim 1, wherein the calculation computing device is further configured to transmit, to the media computing device, a digital media transmission confirmation message to the at least one presentation computing device, which is mobile, said transmission confirmation message including an identifier associated with said presentation computing device and an identifier of the transmitted digital media.

17. The digital media distribution system according to claim 1, wherein the calculation computing device is further configured to transmit, to the media computing device, a message of connection to the at least one presentation computing device, which is mobile, said connection message including an identifier associated with said at least one presentation computing device and an identifier associated with a said shoe parameter value.

18. A method for distributing digital media according to distribution rules, wherein said digital media are distributed to at least one presentation computing device associated with at least one connected sole configured to generate raw use data, said at least one connected sole being associated with a footwear item, said method including the following steps executed by one or more calculation computing devices:
Obtaining mobility or posture parameter values calculated from raw use data;
Obtaining shoe parameter values, said shoe parameter values relating to the footwear item associated with the at least one connected sole;
Obtaining the digital media and a distribution repository including the digital media distribution rules; and
Transmitting personalized digital media to the at least one presentation computing device associated with the at least one connected sole having generated said raw use data from which the mobility or posture parameter values have been generated, said transmitted personalized digital media being selected, within the digital media, according to the distribution rules, the mobility or posture parameter values and the shoe parameter values of the footwear item associated with the at least one connected sole.
